(19) United States
(12) Patent Application Publication
(10) Pub. No.: US 2011/0313466 A1
(43) Pub. Date: Dec. 22, 2011

(54) Spinal Facet Bone Screw System

(76) Inventors: Michael S. Butler, St. Charles, IL (US); Daniel Predick, Chicago, IL (US)

(21) Appl. No.: 13/163,229

(22) Filed: Jun. 17, 2011

Related U.S. Application Data
(60) Provisional application No. 61/355,905, filed on Jun. 17, 2010.

(51) Int. Cl. A61B 17/88; A61B 17/86

(52) U.S. Cl. 606/279; 606/301; 606/305

(57) ABSTRACT

A spinal facet bone screw system provides tactile feedback to the user during installation to aid in determining when one or more components of the spinal facet bone screw system have been installed or implanted. The spinal facet bone screw system has a spinal facet screw and a washer. Tactile installation feedback is provided at an interface between a friction surface of the head of the spinal facet screw and a friction surface of the pocket of the washer to determine when the spinal facet screw has reached its full insertion depth. The bottom or undersurface of the washer that abuts a spinal facet bone or other spinal component during installation may be configured and/or textured to further provide tactile installation feedback as between the washer and the bone or other spinal component.

### RELATED APPLICATIONS

This patent application claims the benefit of and/or priority under 35 U.S.C. §119(e) to U.S. Provisional Patent Application Ser. No, 61/355,905 filed Jun. 17, 2010, entitled “Spinal Facet Fixation System” the entire contents of which is specifically incorporated herein by this reference.

### BACKGROUND OF THE INVENTION

1. Field of the Invention

The present invention relates to devices for the spine and, more particularly, to devices for restricting, limiting and/or stopping motion between adjacent vertebrae.

2. Background Information

As we age various changes can occur in the body especially with respect to the spinal column or spine. For instance, the ligaments of the spine can thicken and calcify (i.e. harden from deposits of calcium), bone and joints may enlarge, bone spurs called osteophytes may form, spinal discs may break down, collapse, bulge and/or herniate. Moreover, one vertebra may slip over another vertebra (spondylolisthesis). Facet joints of the spine can also be affected. The facet joints are found at every spinal level (except at the top level) and provide about 20% of the torsional (twisting) stability in the neck and lower back.

Facet joints are hinge-like and link vertebrae together. They are located at the back or posterior of the spine. Each vertebra has two sets or pairs of facet joint portions that join with two sets or pairs of facet joint portions of adjacent vertebrae. Particularly each vertebra has a pair of upward facing facet joint portions (the superior articular facets) and a pair of downward facing facet joint portions (the inferior articular facets). The pair of superior articular facets of one vertebra is linked to a pair of inferior articular facets of a superiorly adjacent (upper) vertebra, while the pair of inferior articular facets of the one vertebra is linked to a pair of superior articular facets of an inferiorly adjacent (lower) vertebra. Thus, each vertebra is linked to an upper adjacent vertebra by two facet joints and to a lower adjacent vertebra by two facet joints.

The facet joints are synovial joints meaning that each joint is surrounded by a capsule of connective tissue and produces a fluid to nourish and lubricate the facet joint. The facet joints slide or glide smoothly (articulate) on each other and therefore both sliding surfaces are coated by a very low friction, moist cartilage. A small sack or capsule surrounds each facet joint and provides a sticky lubricant for the joint. Each sack has a rich supply of tiny nerve fibers that provide a warning when irritated. The facet joints allow flexion (bend forward), extension (bend backward), and twisting motion, while restricting certain types of movement. The spine is made stable due to the interlocking nature of adjacent vertebrae through the facet joints. Each facet joint is positioned at each level to provide the needed limits to motion, especially to rotation and to prevent forward slipping (spondylolisthesis) of that vertebra over the one below,

Facet joints are in almost constant motion with the spine and quite commonly simply wear out or become degenerated in many people. When facet joints become worn or torn the cartilage may become thin or disappear and there may be a reaction of the bone of the joint underneath producing overgrowth of bone spurs and an enlargement of the joints. The joint is then said to have arthritic (literally, joint inflammation-degeneration) changes, or osteoarthritis that can produce considerable back pain on motion. This condition may also be referred to as “facet joint disease” or “facet joint syndrome”.

Moreover, facet joints become especially vulnerable when the intervertebral disc is injured. This is true because as the disc degenerates, the volume of the disc is reduced and the disc space tends to flatten out. The disc therefore loses height and the space between the vertebral bodies becomes narrowed. The flattening disc space provides much less support for the facet joints at that particular level, and the joints become subject to increased stress. Facet joint pain is often felt in the spinal area rather than in an arm or leg. Other difficulties such as spondylolisthesis and pseudoarthrosis can occur that also causes facet joint problems.

It is therefore sometimes desirable to restrict, limit or immobilize a spinal facet joint because of such disease, injury, trauma or the like. It is may also be desirable to restrict, limit or immobilize a facet joint in order to help stabilize the spine as an aid to spinal fusion.

In view of the above it would thus be desirable to have a fixation device and/or system for restricting, limiting and/or immobilizing a spinal facet joint. More particularly, it would thus be desirable to have a fixation device and/or system for restricting, limiting and/or immobilizing a spinal facet joint that provides an installer to easily and confidently implant the fixation device/system. Moreover, it would be thus desirable to have a fixation device and/or system for restricting, limiting and/or immobilizing a spinal facet joint that indicates to an installer when the fixation device is properly implanted.

### SUMMARY OF THE INVENTION

The present invention is a spinal facet bone screw system which, during installation, provides tactile feedback to the user for determining when one or more components of the spinal facet bone screw system have been fully installed or implanted.

The spinal facet bone screw system has a spinal facet screw component (spinal facet screw) and a washer component (washer). Tactile feedback is provided at or via an interface between a head of the spinal facet screw and a pocket of the washer to determine when the spinal facet screw has reached its full insertion depth.

In order to implement the above, an exemplary embodiment of the present spinal facet bone screw system provides the spinal facet screw head with a convex, radially curved undersurface or bottom. The washer has a concave, radially curved uppersurface, top or pocket that interfaces with the undersurface of the screw head to allow angulation of the washer about the radial undersurface of the screw head. The bottom or undersurface of the washer abuts a spinal facet bone (e.g. a superior articular process) during installation and is configured to provide an appropriate interface between the washer and the bone. In accordance with the present principles, the tactile installation feedback is provided by the interface between the screw head and washer and/or between the washer and bone.

Such optimization of the features of the present spinal facet bone screw system includes having alternate surface finishes, textures, roughening, coating, overspray or the like (surface finishes) on interfacing components to increase the friction between the components so that as the facet screw is inserted into the bone and then interfaces with the washer there will be increased friction that will be detectable by the user. However, the interface between the components of the present spinal facet bone screw system may not be limited to surface finishes and may include features such as teeth that increase friction or inhibit further rotation. Of course, other manners of providing the present tactile installation feedback and/or component optimization may be provided.

In all cases, the present spinal facet bone screw system is made from a biocompatible material such as titanium, stainless steel or other hardened metal and provides active compression of the facet joint.

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a top isometric view of an embodiment of a spinal facet bone screw system fashioned in accordance with the present principles;

FIG. 2 is a side isometric view of the spinal facet bone screw system of FIG. 1;

FIG. 3 is a bottom isometric view of the spinal facet bone screw system of FIG. 1;

FIG. 4 is a side isometric view of the spinal facet bone screw of the spinal facet bone screw system of FIG. 1;

FIG. 5 is a side isometric view of the washer of the spinal facet bone screw system of FIG. 1;

FIG. 6 is a top plan view of the washer of FIG. 5;

FIG. 7 is a bottom plan view of the washer of FIG. 5;

FIG. 8 is a side sectional view of the spinal facet bone screw system of FIG. 1;

FIG. 9 is a bottom isometric view of an upper portion of a spinal facet bone screw of the present spinal facet bone screw system;

FIG. 10 is a top isometric view of a washer for the spinal facet bone screw of FIG. 9 of the present spinal facet bone screw system;

FIG. 11 is a side view of a set of spinal facet bone screw systems fashioned in accordance with the present principles; and

FIG. 12 is a side isometric view of a spinal facet bone screw system of the present invention implanted through a spinal facet joint and associated vertebra of a spine.

Like reference numerals indicate the same or similar parts throughout the several figures.

A discussion of the features, functions and/or configurations of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF THE INVENTION

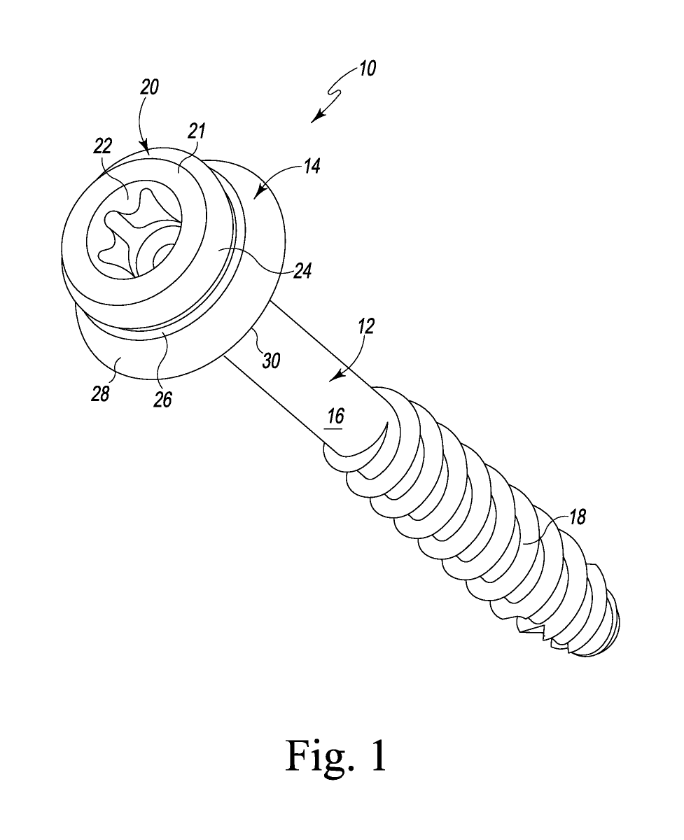

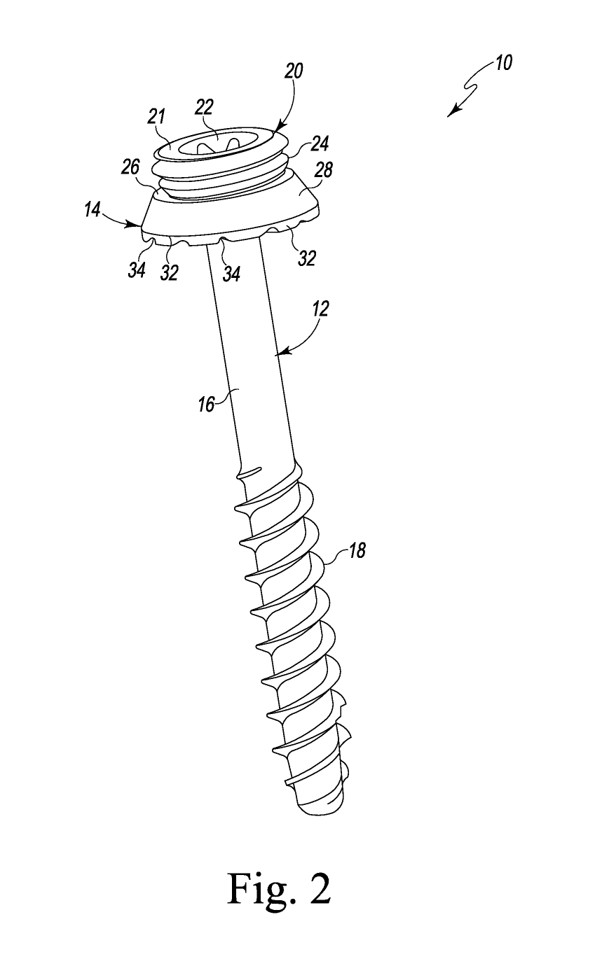

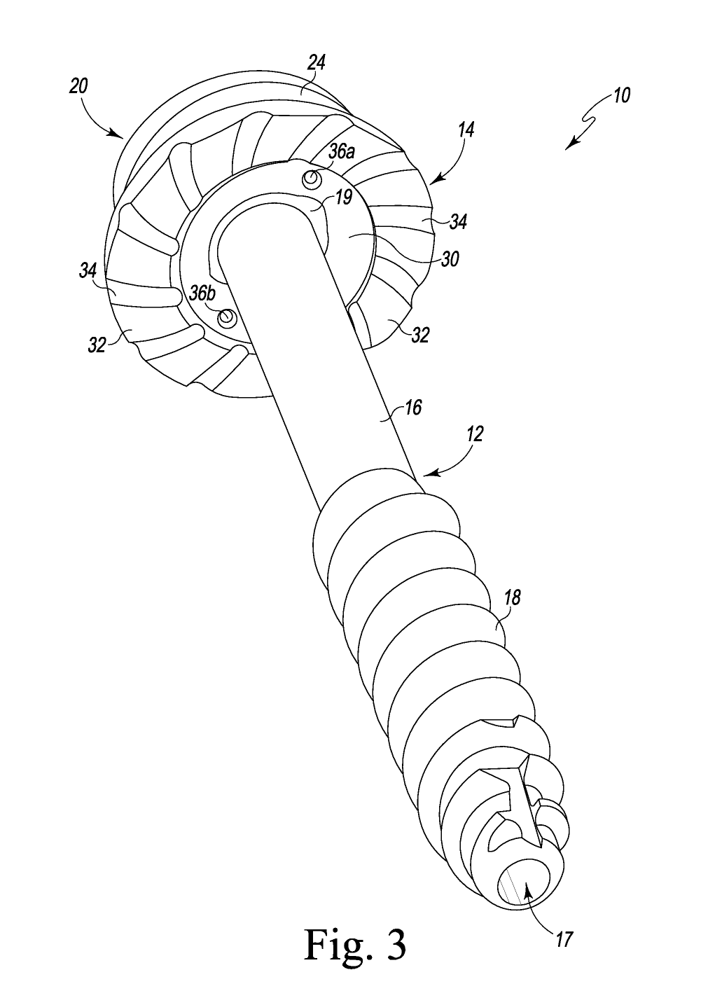

Referring to FIGS. 1-3 there is shown various views of an embodiment of a spinal facet fixation system generally designated 10 fashioned in accordance with the principles of the present invention. The spinal facet fixation system may alternatively be named a spinal facet screw system but will hereinafter be termed a spinal facet fixation system 10. The spinal facet fixation system 10 has a facet screw component (facet screw) 12 and a washer component (washer) 14. The components 12 and 14 are fabricated from a biocompatible material such as titanium, stainless steel or otherwise.

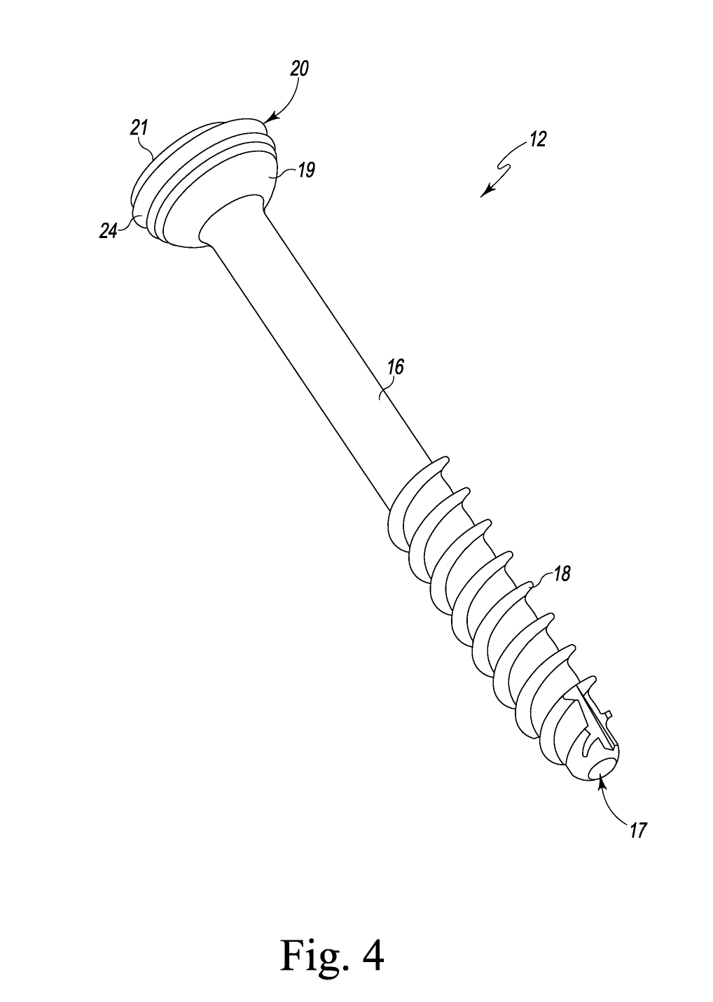

As particularly shown in FIG. 4, the facet screw 12 is formed by a head 20 with an elongated shaft 16 extending from an underside or undersurface 19 of the head 20. The shaft 16 has threads 18 on an outside surface thereof (external threads 18) that extend from an end or tip of the shaft 16 to about halfway up toward the head 20. The shaft 16 may have other thread variations. Additionally, the external threads 18 may or may not be cancellous.

The head 20 is generally annular in configuration and includes threads 24 on an outside annular surface thereof (external threads 24) that extend from a top surface 21 of the head 20 to about halfway down its axial length. The underside 19 is a convex, radially curved surface from which the shaft 16 extends. The head 20 includes a socket 22 that is configured to receive a facet screw driver. The socket 22 is shown having a hex configuration. However, other configurations are contemplated. An elongated axial bore 17 extends through the shaft 16 from the socket 22 to the axial tip of the shaft.

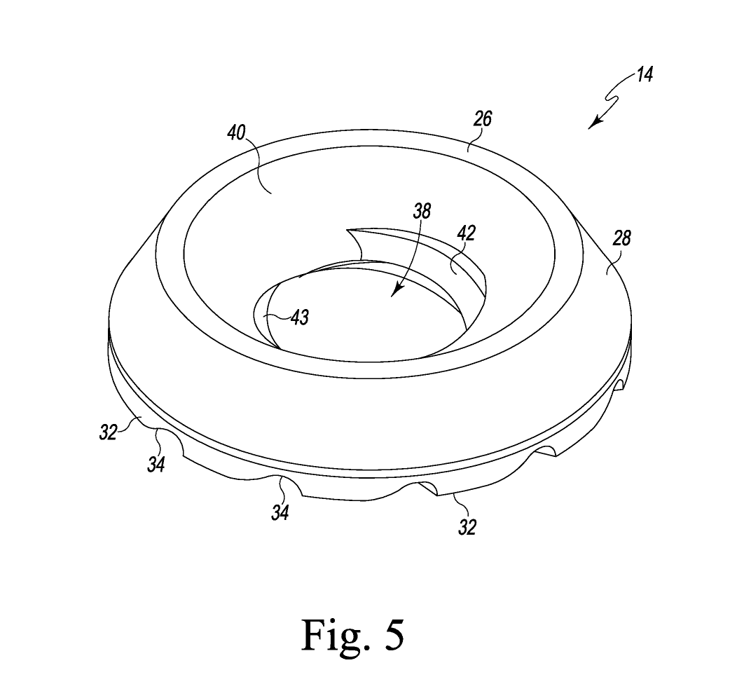

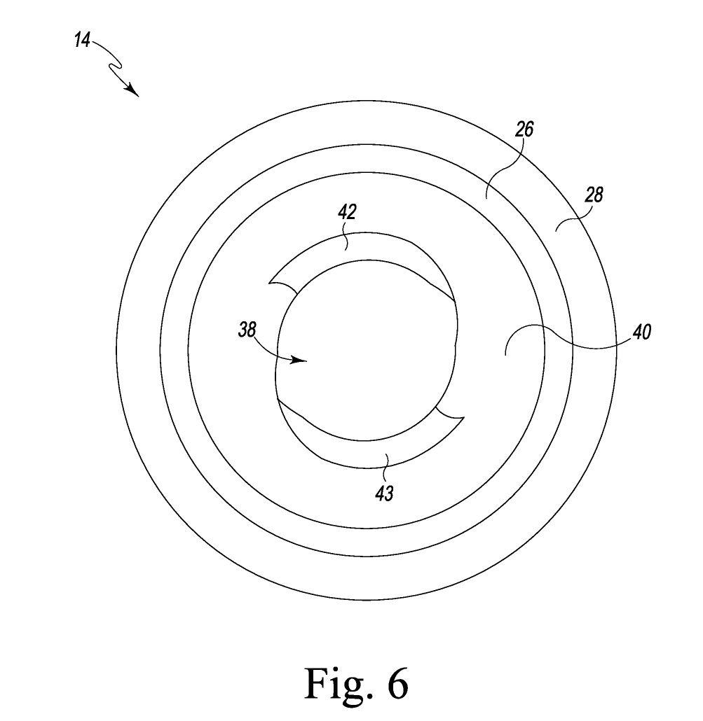

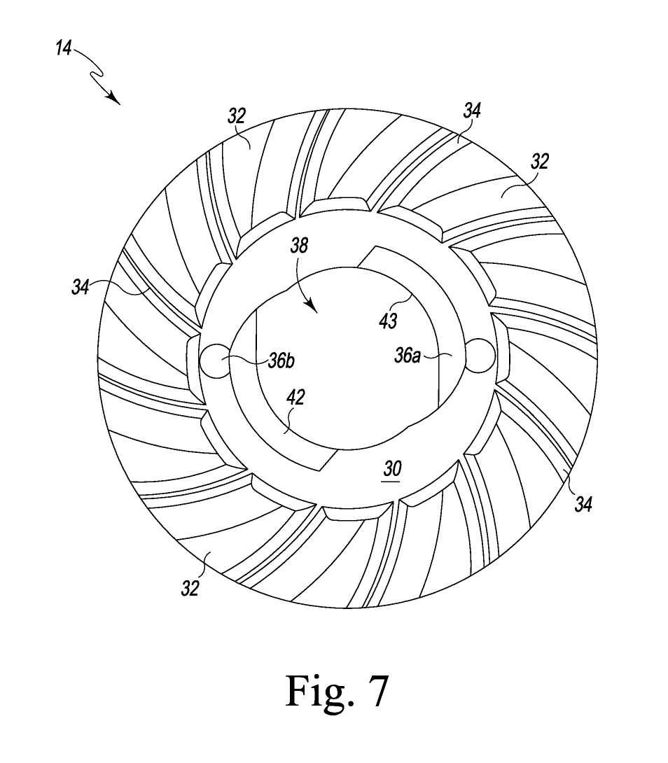

As particularly depicted in FIGS. 5-7, the washer 14 is generally annular in shape and defines a flat, annular top surface 26. An annular side surface 28 extends downwardly and radially outwardly in a tapering, slanting or angled manner. A bore 38 is provided in and through the washer 14. The bore 38 defines a concave uppersurface or upper side/sidewall 40 that extends downwardly and radially inwardly from the top surface 26 in a tapering, slanting or angle manner. The washer 14 also defines an undersurface or bottom 30 having a plurality of radially extending and curved serrated teeth 32. Radially extending and curved channels, slots or notches 34 are defined between the serrated teeth 32. As the washer rotates during installation, the teeth provide gripping into the vertebral bone surface. Additionally, the bottom 30 has two indentions 36a, 36b that are disposed adjacent the bore 38 and the teeth/channels 34. The indentions 36a, 36b are disposed radially opposite one another.

The washer 14 further includes a first arcuate notch 42 disposed at a side of the bore 38 and particularly at the junction of the upper sidewall 40 and the bottom 30 at the bore 38. A second arcuate notch 43 is disposed at another side of the bore 38 and particularly at the junction of the upper sidewall 40 and the bottom 30 at the bore 38. The first and second arcuate notches 42, 43 allow angulation of the screw head 20 when seating in the washer 14.

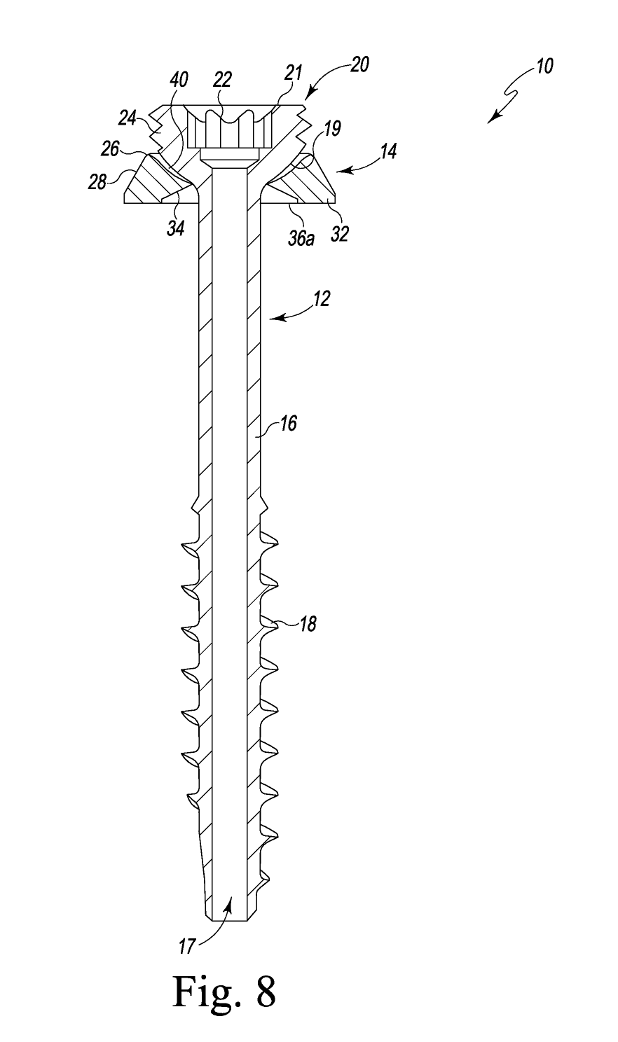

FIG. 8 depicts a side sectional view of the spinal facet fixation system 10. This view provides an illustration of how the curvature of the underside 19 of the head 20 of the facet screw 12 fits into the curvature of the upper surface 40 of the washer 14. It can be appreciated from this view that the head 20 of the facet screw 12, and thus the facet screw 12 itself, is able to angulate with respect to the washer 14.

In accordance with an aspect of the present invention, the underside 19 of the head 20 of the facet screw 12 may be textured or have a surface or surface coating/finish of a particular coefficient of friction while the washer likewise may be textured or have a surface or surface coating/finish of a particular coefficient of friction. The two friction surfaces 19, 40 provide a component interface that increases the friction between the components so that as the facet screw 12 is inserted into the vertebral bone and interfaces with the washer 14, there will be increased friction that is detectable by the user. This provides a tactile feedback during installation. Alternatively to surface finishes, the component interface may include features such as teeth that increase friction or inhibit further rotation.

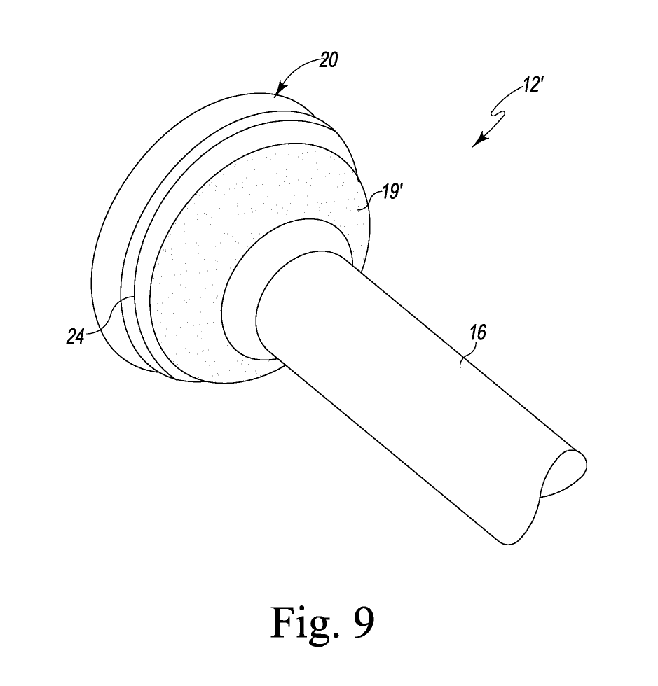

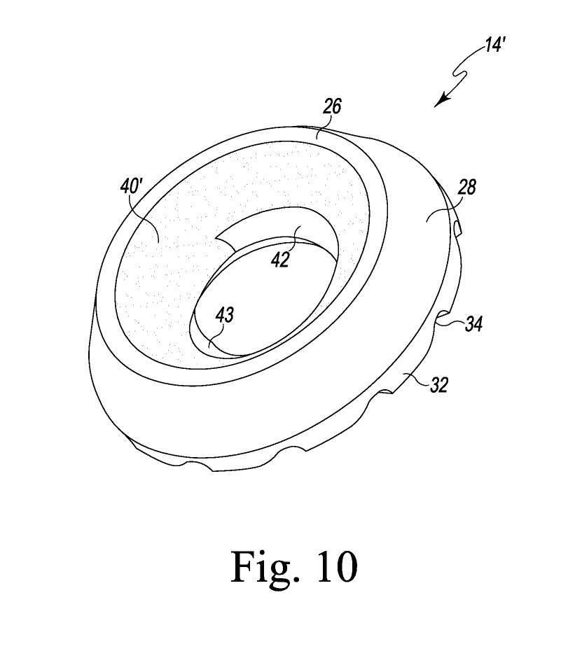

A particular embodiment of this is provided in FIGS. 9 and 10 wherein the under surface 19′ of the spherical head 20 of the bone screw 12′ has a roughened (friction) surface and the spherical washer pocket surface 40′ of the washer 14′ has a like roughened (friction) surface. The roughened portion 19′ and the roughened portion 40′ interface to provide tactile feedback to the surgeon related to depth insertion due to added friction as they compress against one another during installation.

The idea is to optimize the interface between the components to create/provide tactile feedback during installation in order to transmit installation information to the user for indicating proper installation of the spinal facet screw system. Thus, as the user implants or installs the present spinal facet screw system the user will be able to detect by tactile feedback when the facet screw has been properly installed.

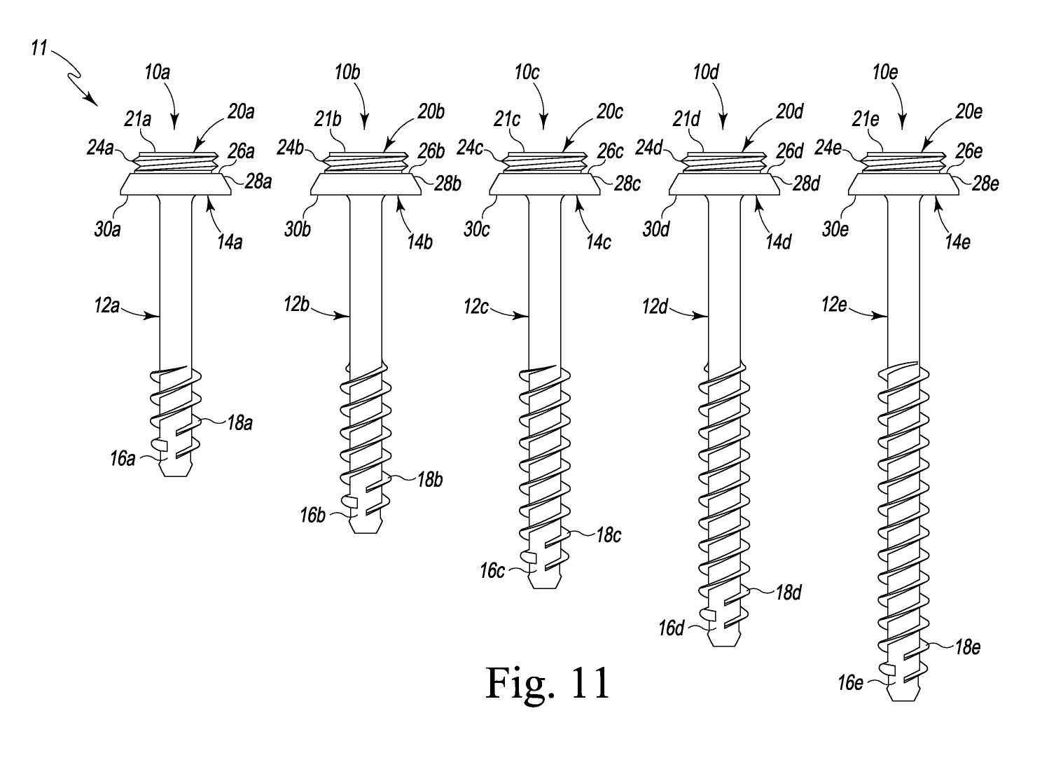

FIG. 11 depicts a set 11 of spinal facet screw fixation systems 10a, 10b, 10c, 10d and 10e. Each spinal facet screw fixation system 10a, 10b, 10c, 10d and 10e includes a facet screw 12a, 12b, 12c, 12d and 12e and a washer 14a, 14b, 14c, 14d and 14e as described above with reference to FIGS. 1-8. Each facet screw 12a, 12b, 12c, 12d and 12e of the set 11 has a different axial or longitudinal length. It can be seen from FIG. 11 that this increase in axial length increases the length of the external threads 18a, 18b, 18c, 18d and 18e. The length between the bottom 30a, 30b, 30c, 30d and 30e of the washers 14a, 14b, 14c, 14d and 14e and the beginning of the external threading 18a, 18b, 18c, 18d and 18e on the shafts 16a, 16b, 16c, 16d and 16e of the facet screws 12a, 12b, 12c, 12d and 12e remains the same. Thus, the longer facet screws allow greater penetration into the vertebral bone. All other dimensions and features of the spinal facet screw fixation systems 10a, 10b, 10c, 10d and 10e remain the same as described above.

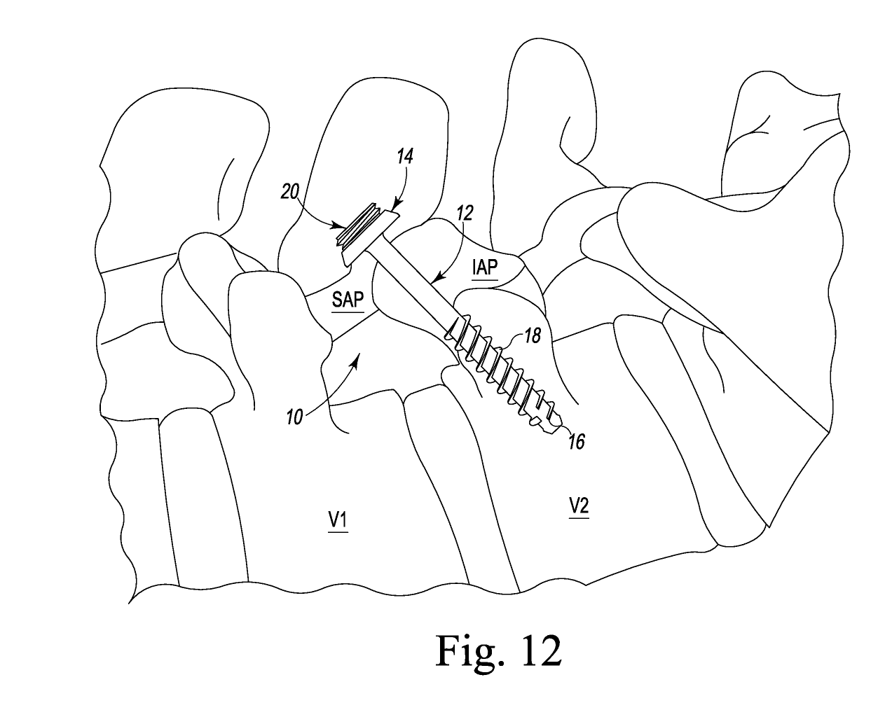

Referring to FIG. 12, there is depicted a spinal facet fixation system 10 installed or implanted into the spine. Particularly, the spinal facet fixation system 10 is installed through a superior articular process SAP of a first vertebra V1 and the inferior articular process IAP of a second, adjacent vertebra V2. The superior articular process SAP of the first vertebra V1 and the inferior articular process IAP of the second, adjacent vertebra V2 for a facet joint that is thus immobilized by the spinal facet fixation system 10. Such immobilization may be accomplished in conjunction with a fusion (not shown) of the vertebrae V1 and V2.

While the invention has been illustrated and described in detail in the drawings and foregoing description, the same is to be considered as illustrative and not restrictive in character, it being understood that only preferred embodiments have been shown and described and that all changes and/or modifications that come within the spirit of the invention are desired to be protected.

What is claimed is:

1. A spinal facet bone screw system comprising:
a spinal facet bone screw having a threaded shank and a head, the head having a spherical underside with a first friction surface; and
a washer having first and second ends, a bore extending through the washer from the first end to the second end, a spherical pocket in the first end, and a second friction surface on the spherical pocket;
the first and second friction surfaces providing tactile feedback in response to an application of torque during installation of the spinal facet bone screw system to aid in determining when the spinal facet bone screw system has reached an implantation depth.

2. The spinal facet bone screw system of claim 1, wherein the first and second friction surfaces each comprise one of a surface finish, texture, roughening, coating or overspray.

3. The spinal facet bone screw system of claim 1, wherein the second end of the washer includes a plurality of teeth.

4. The spinal facet bone screw system of claim 3, wherein the plurality of teeth extend from the bore radially outwardly and arched to an edge of the second end.

5. The spinal facet bone screw system of claim 1, wherein the second end of the washer includes a third friction surface.

6. The spinal facet bone screw system of claim 5, wherein the third friction surface comprises one of a surface finish, texture, roughening, coating or overspray.

7. The spinal facet bone screw system of claim 5, wherein the second end of the washer includes serrations.

8. The spinal facet bone screw system of claim 7, wherein the serrations extend from the bore radially outwardly and arched to an edge of the second end.

9. A spinal implant for immobilizing a spinal facet joint between adjacent vertebrae, the spinal implant comprising:
a spinal facet bone screw having a threaded shank and a head, the head having a spherical undersurface and a drive socket in an upper end thereof; and
a washer having first and second ends, a bore extending through the washer from the first end to the second end, a spherical pocket in the first end, a second friction surface on the spherical pocket, and a third friction surface on the second end;
the first, second and third friction surfaces providing tactile feedback in response to an application of torque during installation of the spinal facet bone screw system to aid in determining when the spinal facet bone screw system has reached an implantation depth.

10. The spinal implant of claim 9, wherein the first, second and third friction surfaces each comprise one of a surface finish, texture, roughening, coating or overspray.

11. The spinal implant of claim 10, wherein the second end of the washer includes serrations.

12. The spinal implant of claim 11, wherein the serrations extend from the bore radially outwardly and arched to an edge of the second end of the washer.

13. The spinal implant of claim 10, wherein the washer further includes a skirt extending from an upper surface of the pocket to the edge of the second end.

14. The spinal implant of claim 13, wherein the spinal facet bone screw and the washer are formed of titanium or stainless steel.

15. A method for immobilizing a spinal facet joint between adjacent vertebrae, the method comprising:
providing a spinal facet bone screw having a threaded shank and a head, the head having a spherical undersurface and a drive socket in an upper end thereof;
providing a washer having first and second ends, a bore extending through the washer from the first end to the second end, a spherical pocket in the first end, a second friction surface on the spherical pocket, and a third friction surface on the second end; and
installing the spinal facet bone screw and washer into bones of a spinal facet joint between adjacent vertebrae by applying torque to the spinal facet bone screw, the first, second and third friction surfaces providing tactile feedback in response to the application of torque to aid in determining when the spinal facet bone screw system has reached an implantation depth.
